(19) United States
(12) Patent Application Publication
(10) Pub. No.: US 2023/0218452 A1
(43) Pub. Date: Jul. 13, 2023

(54) NONWOVEN FABRIC STRUCTURE FOR ABSORBENT ARTICLES AND ABSORBENT ARTICLE COMPRISING SUCH A NONWOVEN FABRIC STRUCTURE

(71) Applicants: PFNONWOVENS HOLDING S.R.O., Prague 1 (CZ); PFNONWOVENS CZECH S.R.O., Znojmo (CZ); PFN - GIC A.S., Znojmo (CZ)

(72) Inventors: Pavlina KASPARKOVA, Znojmo (CZ); Zdenek MECL, Nový Saldorf-Sedlesovice (CZ)

(21) Appl. No.: 17/924,207

(22) PCT Filed: May 10, 2021
(86) PCT No.: PCT/CZ2021/050050

§ 371 Date: Nov. 9, 2022

(30) Foreign Application Priority Data
May 9, 2020 (CZ) PV 2020-257

(51) Int. Cl. A61F 13/534; D04H 1/407; D04H 3/147; D04H 3/018; D04H 3/011; D04H 3/007; D04H 1/28

(52) U.S. Cl. CPC A61F 13/534; D04H 1/407; D04H 3/147; D04H 3/018; D04H 3/011; D04H 3/007; D04H 1/28; A61F 2013/530153

(57) ABSTRACT

A nonwoven fabric structure for absorbent articles comprising a first layer, a second layer and superabsorbent particles, said first layer comprising endless filaments, —which comprise at least a first polymeric material (A) and a second polymeric material (B) having its melting point lower than the first polymeric material (A), —wherein the second polymeric material (B) extends in the longitudinal direction of the filament and forms at least a part of the surface of the filament and—the first layer contains filament-to-filament bonds formed of the second polymeric material (B), —void volume between the filaments of the first layer forms at least 65% of the volume of the first layer, the second layer comprising endless filaments, wherein the superabsorbent particles are arranged at least between the first layer and the second layer and within some of the voids of the first layer and/or the second layer.

### FIELD OF ART

The invention relates to a nonwoven fabric structure for absorbent articles, the structure comprising two layers of endless filaments. The invention also relates to an absorbent article comprising such a nonwoven fabric structure.

### BACKGROUND OF THE INVENTION

Absorbent articles, such as disposable baby diapers, baby diaper pants, underwear, briefs, pants and pads intended for adults suffering from incontinence and female hygiene products, usually contain a backsheet, a topsheet, and an absorbent core arranged between the topsheet and the backsheet and in certain cases there is also an acquisition-distribution layer (ADL) arranged between the topsheet and the core.

The absorbent core may comprise a core wrap within which pulp and superabsorbent particles are arranged.

Alternatively, the core may comprise a core wrap, within which a carded nonwoven fabric with incorporated superabsorbent particles is arranged. The core wrap is provided with an adhesive on the inside, or even within the core itself to immobilize at least some of the superabsorbent particles and to improve the integrity of the core when in use.

It is desirable that such absorbent articles are adapted to receive a gush of liquid, to guide the liquid as quickly as possible into the article in order to get the upper layer of the article (the layer facing the skin of the wearer) dry as soon as possible, i.e. to retain the liquid within the article and to prevent rewetting of the upper skin-contact layer.

It is known that it takes a certain time for the liquid to be absorbed by the superabsorbent particles. Therefore, the risk of leakage shortly after the insult (excretion of body wastes) is high, especially when the wearer moves and compresses the article.

The aim of the invention is to provide a nonwovens fabric structure provided with superabsorbent particles and intended for absorbent articles, which is light-weight and, when arranged within an absorbent article, enables rapid liquid intake and achieves very low rewetting of the outer surface of the absorbent article even when the absorbent article is exposed to pressure shortly after the intake, i.e. even before the liquid is absorbed by the superabsorbent and/or pulp. In other words, the novel nonwoven structure should act as a temporary containment medium for the fluid discharged into the hygiene article, where this containment medium then subsequently empties itself relatively slowly as the superabsorbent gradually binds the fluid.

The nonwoven fabric structure shall be bulky and soft, so that when pressure is applied (for example, when the wearer sits on it) it shall be compressed smoothly. And the nonwoven fabric structure shall also recover when the pressure is released.

### SUMMARY OF THE INVENTION

The above mentioned and some other drawbacks of prior art are eliminated by the nonwoven fabric structure defined in claim 1 and comprising a first layer, a second layer and superabsorbent particles, said first layer comprising endless filaments,
which comprise at least a first polymeric material (A) and a second polymeric material (B) having its melting point lower than the first polymeric material (A),
wherein the second polymeric material (B) extends in the longitudinal direction of the filament and forms at least a part of the surface of the filament and
the first layer contains filament-to-filament bonds formed of the second polymeric material (B),
calculated void volume between the filaments of the first layer forms at least 65% of the volume of the first layer, wherein the calculated void volume between the filaments of the first layer=[1−(volume of the mass of filaments in 1 m² of the first layer/volume of 1 m² of the first layer)]*100%, and the second layer comprising endless filaments,
   wherein the superabsorbent particles are arranged at least between the first layer and the second layer and within some of the voids of the first layer and/or the second layer.

All components of the filaments of the first layer may be arranged across the cross-section of the filament in a configuration of supporting crimping or in a crimping supporting configuration.

Preferably:
at least 20% of the filaments of the first layer have a length of filament to length of fabric ratio higher than 1.2:1, and/or
at least 10% of the filaments of the first layer have a ‘length of filament to length of fabric ratio’ higher than 1.5:1, and/or
at least 10% of the filaments of the first layer have a ‘length of filament to length of fabric ratio’ lower than 2.5:1.

More preferably, the void volume between the filaments of the first layer forms at least 75% of the volume of the first layer preferably at least 80%; more preferably at least 84%, more preferably at least 86%, more preferably of at least 88%, more preferably at least 90%, most preferably at least 93% of the volume of the first layer.

It is also advantageous, when the first polymeric material (A) and/or the second polymeric material (B) of the filaments of the first layer consists of or comprises as the majority component a polymeric material selected from the group consisting of polyesters, polyolefins, polylactic acid, polyester copolymers, polylactide copolymers and blends thereof; and the first polymeric material (A) is different from the second polymeric material (B).

According to an advantageous embodiment, the filaments of the first layer and or of the second layer have a core/sheath structure, wherein the first polymeric material (A) forms the core and the second polymeric material (B) forms the sheath.

Preferably, the first layer and/or the second layer has a basis weight of at least 5 gsm, preferably of at least 10 gsm, more preferably of at least 20 gsm, more preferably of at least 30 gsm, with advantage of at least 40 gsm and preferably not greater than 200 gsm, preferably not greater than 150 gsm, preferably not greater than 100 gsm, most preferably not greater than 80 gsm.

Also preferably, the filaments of the first layer and/or of the second layer have a median fibre diameter of at least 5 microns; preferably at least 10 microns; preferably at least 15 microns; most preferably at least 20 microns, and at most 50 microns; preferably at most 40 microns; most preferably at most 35 microns. According to a further advantageous embodiment
the endless filaments of the second layer comprise at least the first polymeric material (A) and the second polymeric material (B), which extends in the longitudinal direction of the filaments and forms at least a part of the surface of the filaments and
the second layer contains filament-to-filament bonds formed by the second polymeric material (B).

In some cases, it is preferred that the filaments of the second layer have an eccentric core/sheath structure. The filaments of the second layer may be crimped filaments exhibiting at least 3 crimps/cm.

Preferably, the nonwoven fabric structure further comprises a third layer of endless filaments arranged adjacent the second layer at the side facing away from the first layer, the endless filaments of the second and third layer being both crimped filaments exhibiting at least 3 crimps/cm.

Most preferably, the filaments of the first layer and the filaments of the second layer have hydrophilic surface.

According to a particularly preferred embodiment the first layer is more hydrophilic than the second layer.

Preferably, the superabsorbent particles are superabsorbent polymer particles, more preferably the superabsorbent particles contain crosslinked, partly neutralized polyacrylic acid and/or sodium polyacrylate.

Also preferably, at least 50% by weight, more preferably at least 65% by weight of the superabsorbent particles has the size within the range of 300 to 850 microns.

Preferably, the nonwoven fabric structure contains at least 200 g, more preferably at least 300 g, more preferably at least 400 g of superabsorbent particles per m2, wherein it contains less than 800 g, more preferably less 700 g, most preferably less than 600 g of superabsorbent particles per m2.

The above described drawbacks of prior art are also eliminated by an absorbent hygienic product comprising a topsheet, a backsheet and the nonwoven fabric structure according to the invention arranged between the topsheet and the backsheet. Preferably, the first layer is arranged closer to the backsheet than the second layer.

### Definitions

The term “batt” refers to materials in the form of staple fibres or filaments that are found in the state prior to bonding, a process that can be performed in various ways, for example, air-through-bonding, calendaring etc. The “batt” consists of individual fibres or filaments between which a fixed mutual bond is usually not yet formed even though the filaments may be pre-bonded/pre-consolidated in certain ways, where this pre-consolidation may occur during or shortly after the laying of the filaments in the spunlaying process. This pre-consolidation, however, still permits a substantial number of the filaments to be freely moveable such that they can be repositioned. The above mentioned “batt” may consist of several strata created by the deposition of filaments from several spinning beams in the spunlaying process.

The term “filament” refers to a principally endless fibre, while the term “staple fibre” refers to a fibre which has been cut to a defined length.

The term “filament-to-filament bonds” refers to bonds which connect usually two filaments in an area, in which the filaments cross each other or locally meet or abut on each other. The bonds may connect more than two filaments or may connect two parts of the same filament.

The term “mono-component filament” refers to a filament formed of a single polymer or polymer blend, as distinguished from a bi-component or multi-component filament.

“Multi-component fibre or filament” refers to a fibre or filament having a cross-section comprising more than one discrete section, where each of these sections comprises a different polymer component, or a different blend of polymer components, or polymer component and blend of polymer components. The term “multi-component fibre/filament” includes, but is not limited to, “bi-component fibre/filament.” The different components of multi-component fibres are arranged in substantially distinct regions across the cross-section of the fibre and extend continuously along the length of the fibre. A multi-component fibre may have an overall cross-section divided into subsections of the differing components of any shape or arrangement, including, for example, coaxial subsections, core-and-sheath subsections, side-by-side subsections, radial subsections, islands-in-the-sea subsections, etc.

A bi-component filament having a “core/sheath structure” has a cross-section comprising two discrete polymer or polymer blend sections, wherein the sheath polymer or polymer blend component is disposed around the core polymer or polymer blend component.

The term “eccentric core/sheath” structure refers to a filament having a cross-section, in which the centre-of-mass of the core component is offset from the centre-of-mass of the filament. When the sheath component has different solidification characteristics than the core component, especially when the sheath component has a melting point at least 20° C. lower than the melting point of the core component, such a structure promotes crimping of the filament.

“Fibre diameter” is expressed in units of micrometre/micron (μm). The terms “grams of fibre per 9000 m” (denier or den) or “grams of fibre per 10000 m” (dTex) are used to describe the fineness or coarseness of fibres, which are related to the diameter (when assumed to be circular) by the density of the employed material(s).

“Film”—means a skin-like or membrane-like layer of material formed of one or more polymers, which does not have a form consisting predominantly of a web-like structure of consolidated polymer fibres and/or other fibres.

“Machine direction” (MD)—with respect to the production of a nonwoven web material and the nonwoven web material, machine direction (MD) refers to the direction along the web material substantially parallel to the direction of forward travel of the web material through the production line on which the web material is manufactured.

“Cross direction” (CD)—with respect to the production of a nonwoven web material and the nonwoven web material, cross direction (CD) refers to the direction along the web material substantially transverse to the direction of forward travel of the web material through the production line on which the web material is manufactured.

A “nonwoven” or “nonwoven fabric” or “nonwoven web” is a manufactured sheet or web of directionally or randomly oriented fibres, which are first formed into a batt and then consolidated together by friction, cohesion, adhesion and bonded thermally (e.g. air-through-bonding, calender-bonding, ultrasonic bonding, etc.), chemically (e.g. using glue or binder), mechanically (e.g. hydro-entanglement, etc.) or by combination thereof. The term does not include fabrics which are woven, knitted, or stitch-bonded with yarns or filaments. The fibres may be of natural or man-made origin and may be staple or continuous filaments or be formed in-situ. Commercially available fibres have diameters ranging from less than about 0.001 mm to more than about 0.2 mm, and come in several different forms: short fibres (known as staple or chopped fibres), continuous single fibres (filaments or monofilaments), untwisted bundles of continuous filaments (tow), and twisted bundles of continuous filaments (yarn). Nonwoven fabrics can be formed by many processes including, but not limited to, meltblowing, spunbonding, spunmelting, solvent spinning, electro-spinning, carding, film fibrillation, melt-film fibrillation, air-laying, dry-laying, wet-laying with staple fibres, and combinations of these processes as known in the art. The basis weight of nonwoven fabrics is usually expressed in grams per square meter (gsm).

The term “absorbent hygiene product/article” herein refers to products or aids that absorb or retain bodily excretions; more specifically to products or aids, that are placed against the body or placed in the vicinity of the body of the user/wearer for the purpose of absorbing and retaining various bodily excretions. Absorbent hygiene products may include disposable diapers, diaper pants, underwear and pads intended for adults suffering from incontinence, female hygiene products, nursing pads, disposable changing pads, bibs, bandages and similar products. The term “excretions” refers to, in the sense used herein, namely to urine, blood, vaginal secretions, breast milk, sweat and faeces.

As used herein, the term “layer” refers to a sub-component or element of a web. A “layer” may be in the form of a plurality of fibres made on a single beam or on two or more consecutive beams, which produce substantially the same fibres. For example, two consecutively arranged spunbond beams with substantially the same settings and polymer compositions can together produce a single layer. In contrast, for example, two spunbond beams, where one produces mono-component fibres and the other bi-component fibres, will form two distinct layers. The composition of a layer can be determined either by knowing the individual settings and components of the resin (polymer) composition used to form the layer, or by analyzing the nonwoven itself, using, for example, optical or SEM microscopy or by analysing the composition used to make the fibres of the layer using DSC or NMR methods. The term “layer” also refers to a pre-bonded fibre layer which can be for example wound on a roll and transported to further processing (e.g. chemical bonding with a superabsorbent and another layer).

The “spunbond” process is a nonwoven manufacturing system involving the direct conversion of a polymer into continuous filaments, integrated with the conversion of the filaments into a random arrangement of laid filaments forming a nonwoven batt that is subsequently bonded to form a nonwoven fabric. The bonding process can be performed in various ways, for example, air-through-bonding, calendaring, etc.

“Activation” herein refers to the process, whereby fibres or filaments or fibre structures being in a semi-stable state (for example not being crystallized in the lowest possible energy state) are heated and then slowly cooled so, that the semi-stable state changes to some other more stable state (for example a different crystallization phase).

The term “crimping supporting cross-section” herein refers to multi-component fibres, where components with different shrinkage properties are arranged across the cross-section so, that when heated to or above the activation temperature and then slowly cooled down, the fibres crimp, which causes these fibres to follow the vectors of the shrinkage forces. Thereby, when the fibre is released, it creates a so-called helical crimp, although when contained within a fibre layer the mutual adhesion of the fibres does not permit the creation of ideal helixes. For a multi-component fibre, we can determine the centre-of-mass for each individual component in the fibre cross-section (considering their areas/positions in the cross-section). Not to be bound by a theory, it is believed that when the centres-of-mass of all areas of each of the components are substantially at the same point, the fibre is non-crimpable. For example, for a round bi-component fibre with a concentric core/sheath structure the centre-of-mass is in the centre of the cross-section. Various possible crimpable cross-sections are shown in FIG. 1, non-crimpable cross-sections of filaments are shown in FIG. 2.

The term “compressibility” herein refers to the distance in millimetres (mm) by which the nonwoven is compressed by a load defined by a “resilience” measurement.

The term “spinneret capillary density [1000/m]” herein refers to the number of capillaries placed on the spinneret per 1 m distance in the Cross Direction.

The term “filament speed” herein refers to a number calculated from the fibre diameter, the throughput and the polymer density of the filament.

The term “draw down ratio” herein refers to a number calculated by dividing the capillary cross-section area by the filament cross-section area. The measured fibre fineness based on its apparent diameter is used to calculate the filament cross-section area. Other non-round cross-sections cannot be calculated in this way, thus in such cases the analysis of SEM images showing the actual cross-section is necessary.

The term “cooling air/polymer ratio” herein refers to a number calculated by dividing cooling air mass flow by the polymer mass flow.

### BRIEF DESCRIPTION OF THE DRAWINGS

Preferred embodiments of the invention will be further described in more detail with reference to the accompanying schematic drawings, which show:

FIG. 1 examples of crimpable cross-section of filaments or fibres,

FIG. 2 examples of non-crimpable cross section of filaments or fibres,

FIG. 3 is a schematic side view of the nonwovens fabric structure,

FIG. 4 is a schematic cross-sectional view of a part of an absorbent article containing the nonwovens fabric structure,

FIG. 5 shows the distribution paths of liquid throughout the nonwoven fabric structure.

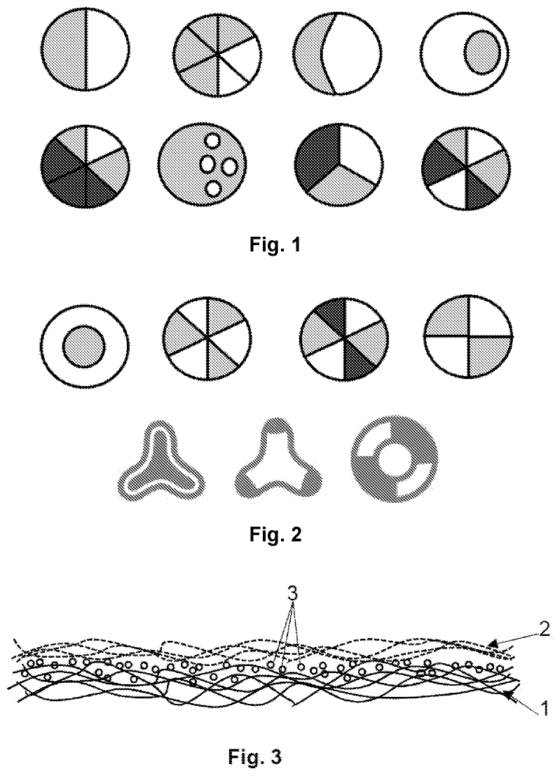

### DETAILED DESCRIPTION OF EXEMPLARY EMBODIMENTS

According to the invention, the nonwoven fabric structure comprises at least a first layer, a second layer and superabsorbent particles arranged between the first layer and the second layer and also partially in the voids within at least one of the layers. The layers are formed mainly from endless filaments. The filaments can be multi-component, preferably bi-component.

In one embodiment according to the invention, the first layer and/or the second layer comprises endless filaments with a non crimpable cross-section. Not to be bound by a theory, it is believed that when the centres-of-mass of surfaces formed by each of the components within the fibre cross-section are located at substantially the same point, the cross-section is considered to be non-crimpable. For example, such a layer of filaments having a non-crimpable cross-section can comprise mainly endless filaments with a round cross-section, trilobal cross-section, star cross-section, etc. A person skilled in the art will understand the many possible shapes of the fibre cross-section that will substantially neither crimp when cooled, nor exhibit latent crimping, which may, however, be activated by heating and subsequent cooling of the fibres.

For example, endless filaments can be multi-component filaments, where the component layout in the cross-section is core/sheath (concentric), segmented pie or any other layout with the centre-of-mass of component areas in one location within the filament cross-section (FIG. 2).

Preferably, such a layer is formed from bi-component core/sheath filaments with a round or trilobal shape.

According to the invention, the endless filaments are formed from two or more components, wherein one component brings a certain level of strength and rigidity that is necessary for the recovery feature and the other component brings softness and also is able to maintain a cohesive structure by forming bonds between the individual filaments.

The first component can be chosen, e.g. from a group of polyesters (e.g. from aromatic polyesters such as polyethylene terephthalate (PET), or from aliphatic polyesters such as polylactic acid (PLA)), polyamides, polyurethanes or their copolymers or suitable blends. It is within the scope of the invention that the first component consists or consists essentially of a plastic of the group of polyesters that also includes polyester copolymers (coPET) or polylactide copolymers (coPLA). Preferably polyethylene terephthalate (PET) or polylactic acid (PLA) is used as the polyester.

The second component can be chosen, e.g. from a group of polyolefins (i.e. polypropylene or polyethylene), low-melting polymers, copolymers or blends of suitable polymers. It is within the scope of the invention that the second component consists or consists essentially of a plastic of the group of polyesters that also includes polyester copolymers (coPET) or polylactide copolymers (COPLA). Preferably polyethylene (PE) is used as the polyolefin.

The preferred combination of components for the bi-component filaments for the first and/or second layer of filaments having non-crimpable cross-sections are PET/PE, PET/PP, PET/CoPET, PLA/COPLA, PLA/PE and PLA/PP.

The preferred bi-component filaments for the first and/or second layer of filaments having non-crimpable cross-sections have the ratio of the mass of the first component to the mass of the second component from 50:50 to 90:10.

In another embodiment, the components of the filaments with a cross-section not supporting crimping within the first layer can also contain additives to modify the filament properties. For example, the core can contain a colour pigment or, for example, a nucleating agent. A person skilled in the art will understand that special combinations of nucleating agents can be found that can change the polymer crystallization and shrinkage behaviour up to a significant level (e.g. as shown by Gajanan in U.S. Pat. No. 5,753,736, filed in 1995). On the other hand, for example, simple titanium dioxide, which is often used as a whitening colouring agent, will cause only an insignificant change in the polymer behaviour that can be, in case of need, easily offset by a slight adjustment of the process conditions. The surface part of the filament, for example sheath, can contain, for example, a colour pigment or a surface modifier (to attain, for example, a silky touch and feel quality).

In another embodiment, the components can also contain a certain amount of further polymers. For example, the first component (e.g. core) can contain a certain small amount of the second component (e.g. sheath) polymer or polymers, or vice versa the second component (e.g. sheath) can contain, for example, a small amount of second component (e.g. core) polymer or polymers.

A person skilled in the art will know that a certain content level can be found for exact polymer combinations. For example, Moore teaches (US application US2012088424 from 3M innovative properties) that a blend of up to 10% of polypropylene to polyester will provide stable fibres.

Preferably, the first layer has a basis weight within the range of 5 to 200 gsm, more preferably within the range of 10 to 100 gsm and most preferably within the range of 30 to 80 gsm.

Also preferably, the filaments of the first layer have a median fibre diameter within the range of 5 to 50 microns, more preferably 10 to 40 microns, most preferably within the range of 15 to 35 microns.

According to an advantageous embodiment, the second layer is substantially the same as the first layer.

According to another preferred embodiment, the first layer and/or the second layer comprises crimped endless filaments, preferably multi-component filaments, most preferably bi-component filaments, having a crimpable cross-section.

Preferably, such a layer is formed from bi-component side-by-side or eccentric core/sheath filaments with a round or trilobal shape.

Preferably, the filaments have a side-by-side or eccentric core/sheath structure, wherein the surface of the fibre (e.g. sheath or one side) comprises a polymer having a lower melting point temperature than the material of the core or second component of the fibre.

According to a preferred embodiment, the higher melting temperature component (core or side) comprises a polymer selected from the group containing polyesters, polyamides and polyolefins, preferably PET, coPET, PLA or PP. It is also advantageous, when the component with the lower melting point (sheath or side) comprises a polymer selected from the group containing polyethylene homopolymer, polyethylene copolymer, polypropylene homopolymer, polypropylene copolymer or polyester copolymer (coPET, coPLA).

In another embodiment, the component of the filaments with a crimpable cross-section can contain additives to modify the crimping. For example, so called nucleating agents are known to improve the crimping level of the filament and thus the bulkiness and possibly also the recovery of the fabric. Nucleating agents may be, for example, aromatic carboxylic acid salts, phosphate ester salts, sodium benzoate, talc and certain pigment colorants, such as for example TiO2. Such a layer of crimped filaments preferably comprises crimped filaments exhibiting at least 3 crimps per centimetre, more preferably 5 to 15 crimps/cm.

According to a further advantageous embodiment, the nonwoven fabric structure further comprises a third layer of filaments, the third layer being arranged adjacent to the second layer at that side of the second layer, which faces away from the first layer.

Preferably, the third layer contains curly crosslinked cellulose fibres. Preferably the diameter of such cellulose fibres is within the range of 15 to 40 microns, more preferably within the range of 20 to 30 microns. According to a preferred embodiment, the third layer of cellulose fibres has a basis weight within the range of 70 to 170 gsm, more preferably 80 to 160 gsm, even more preferably 90 to 150 gsm, most preferably within the range of 100 to 140 gsm.

Preferably the superabsorbent particles are superabsorbent polymer particles, more preferably containing or consisting of crosslinked, partly neutralised polyacrylic acid and/or sodium polyacrylate. The particle size of the superabsorbent particles is preferably such that at least 50% by weight, more preferably at least 65% by weight are within the range of 300 to 850 microns, when measured by means of sieve analysis (e.g. passing the particles successively through 20, 30, 50, 100, 325 mesh sieves).

Preferably, the nonwovens fabric structure contains about 200 to 800 g of superabsorbent particles per m2 of the nonwoven fabric structure, more preferably 300 g to 700, even more preferably 400 g to 600 g, and most preferably 450 to 550 g of superabsorbent particles per m2 of the nonwoven fabric structure. In a typical baby diaper in maxi (toddler) size, the absorbent core will have dimensions of about 10 cm width and 30 cm length, which translates to approx. 15 g superabsorber per diaper.

The first layer itself, and preferably the second layer also, shall be lofty and bulky, which can be described by layer thickness. The layers shall not be tough; they shall be pleasant and comfortable for the final user, they shall provide a soft-loft. Thus when pressure is applied (for example, when sitting) they shall smoothly compress under low pressure. This can be described by compressibility in a length unit (e.g. mm). The layers shall also recover when freed from the pressure, i.e. what can be described by a recovery measurement. The balance of all the above-mentioned properties can be expressed by Structural softness for each of the first layer and the second layer.

$$Structural softness = ( thickness / basis weight ) ⋆ recovery * ( compressibility / basis weight ) ⋆ 10 e 6 \\ Structural softness = \frac{thickness}{basis weight} \times recovery \times \frac{compressibility}{basis weight} \times 1 {0}^{6}$$

Thickness is in millimetres (mm)

Basis weight is in grams per square meter (gsm)

Recovery is a ratio without a unit

Compressibility in millimetres (mm)=compressibility(ratio without a unit)*thickness (mm)

The first layer (and preferably also the second layer) has a Structural softness of at least 40 m⁴ mm² g⁻²; preferably of at least 80 m⁴ mm² g⁻²; preferably of at least 100 m⁴ mm² g⁻², preferably of at least 110 m⁴ mm² g⁻², more preferably of at least 120 m⁴ mm² g⁻², more preferably of at least 130 m⁴ mm² g⁻², more preferably of at least 140 m⁴ mm² g⁻², with advantage of at least 150 m⁴ mm² g⁻²

The first layer (and preferably also the second layer) has a thickness relative to basis weight (thickness recalculated to 1 gsm=thickness (mm)/basis weight (gsm)) of at least 5e10-3, preferably of at least 10e10-3, more preferably of at least 12e10-3.

The first layer (and preferably also the second layer) has a recovery of at least 0.8 (which corresponds to 80% recovery of the original thickness), preferably of at least 0.82, more preferably of at least 0.84, most preferably of at least 0.85. The first layer (and preferably also the second layer) has a compressibility for each 1 gsm of layer basis weight of at least 0.25 microns (0.00025 mm), preferably of at least 0.75 microns (0.00075 mm), preferably of at least 1.25 microns (0.00125 mm), more preferably of at least 1.75 microns (0.00175 mm). So, for example, a 100 gsm layer has the compressibility of at least 25 microns (0.025 mm), preferably at least 75 microns (0.075 mm), preferably at least 125 microns (0.125 mm), more preferably at least 175 microns (0.175 mm).

The first layer (and preferably also the second layer) has a resilience of at least 5%, preferably of at least 8%, more preferably of at least 10%, more preferably of at least 13%, more preferably of at least 15%.

The method of production of such layers of endless filaments having a non-crimpable cross-section and exhibiting the above defined preferred properties (structural softness, resilience, compressibility) has been described e.g. in the Czech patent application No. PV2018-647.

Method of production of bulky layers of endless filaments having a crimpable cross-section has been disclosed e.g. in WO2018059610.

Such a nonwoven fabric structure according to the invention
a first layer of soft bulky lofty layer of endless filaments with high volume of voids between the filaments,
a second layer of soft bulky lofty layer of endless filaments with high volume of voids between the filaments
and superabsorbent particles contained between the first layer and the second layer and in some of the voids of the first layer and/or the second layer
   may be used as an excellent core within an absorbent article. The superabsorbent particles are entrapped between the layers but partially also within the voids of the layers in a way that they do not form any barrier for a liquid entering the absorbent article. After a first strike (insult) of a liquid, the liquid gushes through the second layer and the first layer in a limited (usually central) location. Due to the high volume of voids within the first and second layer, and due to the discontinuous and finely particulate character of the superabsorbent, most of the liquid gets through the limited area of the layers directly to the backsheet and then spreads in all directions along the backsheet and only then starts to rise through the first layer in the direction towards the second layer, i.e. towards the majority of the superabsorbent particles, which then absorb the liquid while swelling (see FIG. 5).

Thus, although the superabsorbent particles absorb the liquid slowly, i.e. not immediately after the liquid strike, the topsheet is dry again in a very short time and the risk of its rewetting is very low.

Hence, the risk of irritation of the skin of the wearer and the risk of leakage of the absorbent article is significantly reduced by this inventive nonwoven fabric structure.

FIG. 3 shows a schematic view of the nonwoven fabric structure according to the invention, comprising a first layer 1 of endless filaments, a second layer 2 of endless filaments and superabsorbent particles 3 arranged between the two layers 1, 2 and partially also within at least the first layer 1.

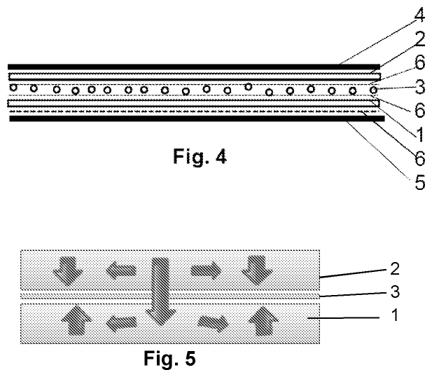

FIG. 4 shows a schematic cross-sectional view of part of an absorbent article (such as e.g. a diaper). The absorbent article comprises a back sheet 5, a layer of glue 6 and the first layer 1 are arranged thereon. The upper side of the first layer 1 may be lightly sprayed with glue. Superabsorbent particles 3 are uniformly distributed on top of the first layer 1 and the second layer 2 is arranged thereon.

Also, the second layer 2 may be provided with a spray of glue on that side, which faces the superabsorbent particles 3 (and the first layer 1). A topsheet 4 is arranged on the second layer 2. In some embodiments of superabsorbent articles, no glue is needed, whilst in other embodiments a spray (drops of, dots, fibre-like particles or any other suitable form) of glue is present at only one of the layers 1, 2 at the side facing the superabsorbent particles, i.e. at the side facing the other layer 2, 1.

### EXAMPLES

A plurality of nonwoven fabric structures according to the invention have been prepared and will be described in more detail below. The plan view (viewed from above in a direction perpendicular to the layers) of each of the nonwovens fabric structure examples was 10×30 cm, wherein the MD direction of the layers extended along the (30 cm) length of the structure). Such nonwovens fabric structures are suitable for use within an absorbent article such as diaper, diaper pants, pads, female hygiene absorbent products, etc.

The spunmelt nonwoven fabric was produced on a REICOFIL technology production line, the fibres were produced using two spunbond beams with the same settings, collected on a belt forming the batt, pre-consolidated and thermobond-consolidated by hot air. As indicated below, some samples were treated with a liquid spin finish on a kiss-roll and dried.

The basis weight of the individual layers (first layer, second layer, third layer) as indicated in the examples below is the basis weight of the filamentary layer as produced not taking into account the weight of the superabsorbent particles.

### Example 1

Example 1 comprised a first layer, a second layer and superabsorbent particles arranged between the first layer and the second layer and some also within the voids of the first or second layer.

The first layer had a basis weight 60 gsm and comprised endless filaments having a concentric core/sheath cross-section, wherein the core consisted of PET (Invista 5520) and formed 70% by weight of the filament and the sheath consisted of coPET (Trevira RT5023) and formed 30% by weight of the filament. The mean filament diameter was 32.5 microns.

The filaments were treated with a spin finish (Silastol PHP 90) to increase their hydrophilicity.

The thickness of the first layer was 1.8 mm, the calculated void volume was 97.57% and the recovery was 99%.

The strike-through time (i.e. the absorption time) of this layer was 1.9 sec. Superabsorbent particles (EVONIC SAP FAVOR SXM 9170) in the amount of 500 g/m², the majority of which have a size within the range of 300 to 850 microns, were evenly distributed on the first layer, and partially within the first layer.

The second layer was the same as the first layer and was placed on the superabsorbent layer.

### Example 2

Example 2 comprised a first layer, a second layer, standard topsheet (15 gsm SB calandred) and superabsorbent particles arranged between the first layer and the second layer and some also within the voids of the first or second layer. The first layer had basis weight 60 gsm and comprised endless filaments having a concentric core/sheath cross section, wherein the core consisted of PET (Invista 5520) and formed 70% by weight of the filament and the sheath consisted of coPET (Trevira RT5023) and formed 30% by weight of the filament. The mean filament diameter was 32.5 microns.

The layer of filaments was treated with spin finish (Silastol PHP 90) to increase their hydrophilicity.

The thickness of the first layer was 1.8 mm, the calculated void volume was 97.57% and the recovery was 99%.

The strike-through time of the first layer was 1.9 sec.

Superabsorbent particles (EVONIC SAP FAVOR SXM 9170) in the amount of 500 g/m², the majority of which have the size within the range of 300 to 850 microns, were evenly distributed on the first layer and partially within the first layer.

The second layer was the same as the first layer and was placed on the superabsorbent layer.

### Example 3

Example 3 comprised a first layer, a second layer and superabsorbent particles arranged between the first layer and the second layer and some also within the voids of the first or second layer.

The first layer had basis weight 60 gsm and comprised endless filaments having a concentric core/sheath cross section, wherein the core consisted of PET (Invista 5520) and formed 70% by weight of the filament and the sheath consisted of coPET (Trevira RT5023) and formed 30% by weight of the filament. The mean filament diameter was 32.5 microns.

The filaments were left untreated.

The thickness of the first layer was 1.8 mm, the calculated void volume was 97.57% and the recovery was 99%.

The strike-through time of the first layer was 3.5 sec.

Superabsorbent particles (EVONIC SAP FAVOR SXM 9170) in the amount of 500 g/m², the majority of which have the size within the range of 300 to 850 microns, were evenly distributed on the first layer and partially within the first layer.

The second layer was the same as the first layer and was placed on the superabsorbent layer.

### Example 4

Example 4 comprised a first layer, a second layer and superabsorbent particles arranged between the first layer and the second layer and some also within the voids of the first or second layer.

The first layer had basis weight 60 gsm and comprised endless filaments having a concentric core/sheath cross section, wherein the core consisted of PET (Invista 5520) and formed 80% by weight of the filament and the sheath consisted of PE (Aspun 6834) and formed

20% by weight of the filament. The mean filament diameter was 23.9 microns. The filaments were treated with spin finish (Silastol PHP 90) to increase their hydrophilicity.

The thickness of the first layer was 1.2 mm, the calculated void volume was 96.13% and the recovery was 98%.

The strike-through time of the first layer was 3.1 sec.

Superabsorbent particles (EVONIC SAP FAVOR SXM 9170) in the amount of 500 g/m², the majority of which have the size within the range of 300 to 850 microns, were evenly distributed between the first layer and the second layer, and partially within the first layer.

The second layer was the same as the first layer and was placed on the superabsorbent layer.

### Example 5

Example 5 comprised a first layer, a second layer and superabsorbent particles arranged between the first layer and the second layer and some also within the voids of the first or second layer.

The first layer had basis weight 80 gsm and comprised endless filaments having a concentric core/sheath cross section, wherein the core consisted of PET (Invista 5520) and formed 70% by weight of the filament and the sheath consisted of coPET (Trevira RT5023) and formed 30% by weight of the filament. The mean filament diameter was 21.9 microns.

The filaments were left untreated.

The thickness of the first layer was 1.62 mm, the calculated void volume was 96.48% and the recovery was 99%.

The strike-through time of the first layer was 5.3 sec.

Superabsorbent particles (EVONIC SAP FAVOR SXM 9170) in the amount of 500 g/m², the majority of which have the size within the range of 300 to 850 microns, were evenly distributed on the first layer and partially within the first layer.

The second layer was the same as the first layer and was placed on the superabsorbent layer.

### Example 6

Example 6 comprised a first layer, a second layer and superabsorbent particles arranged between the first layer and the second layer and some also within the voids of the first or second layer.

The first layer had basis weight 40 gsm and comprised endless filaments having a concentric core/sheath cross section, wherein the core consisted of PET (Invista 5520) and formed 70% by weight of the filament and the sheath consisted of coPET (Trevira RT5023) and formed 30% by weight of the filament. The mean filament diameter was 23 microns.

The filaments were left untreated.

The thickness of the first layer was 1.34 mm, the calculated void volume was 97.79% and the recovery was 97%.

The strike-through time of the first layer was 2.4 sec.

Superabsorbent particles (EVONIC SAP FAVOR SXM 9170) in the amount of 500 g/m², the majority of which have the size within the range of 300 to 850 microns, were evenly distributed on the first layer and partially within the first layer.

The second layer was the same as the first layer and was placed on the superabsorbent layer.

### Example 7

Example 7 comprised a first layer, a second layer and superabsorbent particles arranged between the first layer and the second layer and some also within the voids of the first or second layer.

The first layer had basis weight 60 gsm and comprised crimped endless filaments having an eccentric core/sheath cross section, wherein the core consisted of PET (Invista 5520) and formed 70% by weight of the filament and the sheath consisted of PE (Aspun 6834) and formed 30% by weight of the filament. The mean filament diameter was 14 microns.

The filaments were treated with spin finish (Silastol PHP 90) to increase their hydrophilicity.

The thickness of the first layer was 1 mm, the calculated void volume was 95.05% and the recovery was 97%.

The strike-through time of the first layer was 3.8 sec.

Superabsorbent particles (EVONIC SAP FAVOR SXM 9170) in the amount of 500 g/m², the majority of which have the size within the range of 300 to 850 microns, were evenly distributed on the first layer and partially within the first layer.

The second layer was the same as the first layer and was playced on the superabsorbent layer.

### Example 8

Example 8 comprised a first layer, a second layer and superabsorbent particles arranged between the first layer and the second layer and some also within the voids of the first or second layer.

The first layer had basis weight 80 gsm and comprised endless filaments having a concentric core/sheath cross section, wherein the core consisted of PET

(Invista 5520) and formed 70% by weight of the filament and the sheath consisted of coPET (Trevira RT5023) and formed 30% by weight of the filament. The mean filament diameter was 21.9 microns.

The filaments were left untreated.

The thickness of the first layer was 1.62 mm, the calculated void volume was 96.48% and the recovery was 99%.

The strike-through time of the first layer was 5.3 sec.

Superabsorbent particles (EVONIC SAP FAVOR SXM 9170) in the amount of 500 g/m², the majority of which have the size within the range of 300 to 850 microns, were evenly distributed on the first layer and partially within the first layer.

The second layer had basis weight 40 gsm and comprised endless filaments having a concentric core/sheath cross section, wherein the core consisted of PET (Invista 5520) and formed 70% by weight of the filament and the sheath consisted of coPET (Trevira RT5023) and formed 30% by weight of the filament. The mean filament diameter was 23 microns.

The filaments were left untreated.

The thickness of the second layer was 1.34 mm and the recovery was 97%.

The strike-through time of the second layer was 2.4 sec.

The second layer was placed on the superabsorbent layer.

### Example 9

Example 9 comprised a first layer, a second layer and superabsorbent particles arranged between the first layer and the second layer and some also within the voids of the first or second layer.

The first layer had basis weight 60 gsm and comprised endless filaments having a concentric core/sheath cross section, wherein the core consisted of PET (Invista 5520) and formed 70% by weight of the filament and the sheath consisted of coPET (Trevira RT5023) and formed 30% by weight of the filament. The mean filament diameter was 32.5 microns.

The filaments were left untreated.

The thickness of the first layer was 1.8 mm, the calculated void volume was 97.57% and the recovery was 99%.

The strike-through time of the first layer was 3.5 sec.

The second layer had basis weight 60 gsm and comprised crimped endless filaments having an eccentric core/sheath cross section, wherein the core consisted of PET (Invista 5520) and formed 70% by weight of the filament and the sheath consisted of PE (Trevira RT5023) and formed 30% by weight of the filament. The mean filament diameter was 14 microns.

The filaments were treated with spin finish (Silastol PHP 90) to increase their hydrophilicity.

The thickness of the second layer was 1 mm, the calculated void volume was 95.05% and the recovery was 97%.

The strike-through time of the second layer was 3.8 sec.

Superabsorbent particles (EVONIC SAP FAVOR SXM 9170) in the amount of 500 g/m², the majority of which have the size within the range of 300 to 850 microns, were evenly distributed on the first layer and partially within the first layer.

The second layer was placed on the superabsorbent layer.

### Example 10

Example 10 comprised a first layer, a second layer, a third layer and superabsorbent particles arranged between the first layer and the second layer and some also within the voids of the first or second layer.

The first layer had basis weight 60 gsm and comprised endless filaments having a concentric core/sheath cross section, wherein the core consisted of PET (Invista 5520) and formed 80% by weight of the filament and the sheath consisted of PE (Aspun 6834) and formed 20% by weight of the filament. The mean filament diameter was 23.9 microns.

The filaments were treated with spin finish (Silastol PHP 90) to increase their hydrophilicity.

The thickness of the first layer was 1.2 mm, the calculated void volume was 96.13% and the recovery was 98%.

The strike-through time of the first layer was 3.1 sec.

The second layer was the same as the first layer.

The strike-through time of the second layer was 3.1 sec.

The third layer had basis weight 120 gsm and comprised curly crosslinked cellulose fibres. The mean fibre diameter was 25 microns.

The fibres were left untreated.

The thickness of the third layer was 2.8 mm and the recovery was 95%.

The strike-through time of the third layer was 2.3 sec.

Superabsorbent particles (EVONIC SAP FAVOR SXM 9170) in the amount of 500 g/m², the majority of which have the size within the range of 300 to 850 microns, were evenly distributed on the first layer and partially within the first

The second layer was placed on the superabsorbent layer.

### Example 11

Example 11 comprised a first layer, a second layer, a third layer and superabsorbent particles arranged between the first layer and the second layer and some also within the voids of the first or second layer.

The first layer had basis weight 60 gsm and comprised endless filaments having a concentric core/sheath cross section, wherein the core consisted of PET (Invista 5520) and formed 80% by weight of the filament and the sheath consisted of PE (Aspun 6834) and formed 20% by weight of the filament. The mean filament diameter was 23.9 microns.

The filaments were treated with spin finish (Silastol PHP 90) to increase their hydrophilicity.

The thickness of the first layer was 1.2 mm, the calculated void volume was 96.13% and the recovery was 98%.

The strike-through time of the first layer was 3.1 sec.

The second layer had basis weight 60 gsm and comprised endless filaments having a concentric core/sheath cross section, wherein the core consisted of PET (Invista 5520) and formed 70% by weight of the filament and the sheath consisted of coPET (Trevira RT5023) and formed 30% by weight of the filament. The mean filament diameter was 32.5 microns.

The filaments were left untreated.

The thickness of the second layer was 1.8 mm, the calculated void volume was 97.57% and the recovery was 99%.

The strike-through time of the second layer was 3.5 sec.

The third layer had basis weight 120 gsm and comprised curly crosslinked cellulose fibres. The mean fibre diameter was 25 microns.

The fibres were left untreated.

The thickness of the third layer was 2.8 mm and the recovery was 95%.

The strike-through time of the third layer was 2.3 sec.

Superabsorbent particles (EVONIC SAP FAVOR SXM 9170) in the amount of 500 g/m², the majority of which have the size within the range of 300 to 850 microns, were evenly distributed on the first layer and partially within the first layer.

The second layer was placed on the superabsorbent layer.

### Example 12

Example 12 comprised a first layer, a second layer, a third layer and superabsorbent particles arranged between the first layer and the second layer and some also within the voids of the first or second layer.

The first layer had basis weight 60 gsm and comprised endless filaments having a concentric core/sheath cross section, wherein the core consisted of PET

(Invista 5520) and formed 80% by weight of the filament and the sheath consisted of PE (Aspun 6834) and formed 20% by weight of the filament. The mean filament diameter was 23.9 microns.

The filaments were left untreated.

The thickness of the first layer was 1.2 mm, the calculated void volume was 96.13% and the recovery was 98%.

The second layer had basis weight 60 gsm and comprised endless filaments having a concentric core/sheath cross section, wherein the core consisted of PET (Invista 5520) and formed 80% by weight of the filament and the sheath consisted of PE (Aspun 6834) and formed 20% by weight of the filament. The mean filament diameter was 23.9 microns.

The filaments were treated with spin finish (Silastol PHP 90) to increase their hydrophilicity.

The thickness of the second layer was 1.2 mm and the recovery was 98%.

The strike-through time of the second layer was 3.1 sec.

The third layer had basis weight 120 gsm and comprised curly crosslinked cellulose fibres. The mean fibre diameter was 25 microns.

The fibres were left untreated.

The thickness of the third layer was 2.8 mm and the recovery was 95%.

The strike-through time of the second layer was 2.3 sec.

Superabsorbent particles (EVONIC SAP FAVOR SXM 9170) in the amount of 500 g/m², the majority of which have the size within the range of 300 to 850 microns, were evenly distributed on the first layer and partially within the first layer.

The second layer was placed on a layer of superabsorbent.

### Example 13

Example 13 comprised a first layer, a second layer, a third layer and superabsorbent particles arranged between the first layer and the second layer and some also within the voids of the first or second layer.

The first layer had basis weight 60 gsm and comprised endless filaments having a concentric core/sheath cross section, wherein the core consisted of PET

(Invista 5520) and formed 70% by weight of the filament and the sheath consisted of coPET (Trevira RT5023) and formed 30% by weight of the filament. The mean filament diameter was 32.5 microns.

The filaments were left untreated.

The thickness of the first layer was 1.8 mm, the calculated void volume was 97.57% and the recovery was 99%.

The strike-through time of the first layer was 3.5 sec.

The second layer was the same as the first layer

The third layer had basis weight 120 gsm and comprised curly crosslinked cellulose fibres. The mean fibre diameter was 25 microns.

The fibres were left untreated.

The thickness of the third layer was 2.8 mm and the recovery was 95%.

The strike-through time of the second layer was 2.3 sec.

Superabsorbent particles (EVONIC SAP FAVOR SXM 9170) in the amount of 500 g/m², the majority of which have the size within the range of 300 to 850 microns, were evenly distributed on the first layer and partially within the first layer.

The second layer was placed on the superabsorbent layer.

### Example 14

Example 14 comprised a first layer, a second layer, a third layer and superabsorbent particles arranged between the first layer and the second layer and some also within the voids of the first or second layer.

The first layer had basis weight 60 gsm and comprised endless filaments having a concentric core/sheath cross section, wherein the core consisted of PET

(Invista 5520) and formed 70% by weight of the filament and the sheath consisted of coPET (Trevira RT5023) and formed 30% by weight of the filament. The mean filament diameter was 32.5 microns.

The filaments were left untreated.

The thickness of the first layer was 1.2 mm, the calculated void volume was 97.57% and the recovery was 98%.

The strike-through time of the first layer was 3.5 sec.

The second layer had basis weight 60 gsm and comprised endless filaments having a concentric core/sheath cross section, wherein the core consisted of PET (Invista 5520) and formed 80% by weight of the filament and the sheath consisted of PE (Aspun 6834) and formed 20% by weight of the filament. The mean filament diameter was 23.9 microns.

The filaments were treated with spin finish (Silastol PHP 90) to increase their hydrophilicity.

The thickness of the second layer was 1.2 mm, the calculated void volume was 96.13% and the recovery was 98%.

The strike-through time of the second layer was 3.1 sec.

The third layer had basis weight 120 gsm and comprised curly crosslinked cellulose fibres. The mean fibre diameter was 25 microns.

The fibres were left untreated.

The thickness of the third layer was 2.8 mm and the recovery was 95%.

The strike-through time of the second layer was 2.3 sec.

Superabsorbent particles (EVONIC SAP FAVOR SXM 9170) in the amount of 500 g/m², the majority of which have the size within the range of 300 to 850 microns, were evenly distributed on the first layer and partially within the first layer.

The second layer was placed on the superabsorbent layer.

### Example 15

Example 15 comprised a first layer, a second layer, a third layer and superabsorbent particles arranged between the first layer and the second layer and some also within the voids of the first or second layer.

The first layer had basis weight 60 gsm and comprised endless filaments having a concentric core/sheath cross section, wherein the core consisted of PET (Invista 5520) and formed 80% by weight of the filament and the sheath consisted of PE (Aspun 6834) and formed 20% by weight of the filament. The mean filament diameter was 23.9 microns.

The filaments were treated with spin finish (Silastol PHP 90) to increase their hydrophilicity.

The thickness of the first layer was 1.2 mm, the calculated void volume was 96.13% and the recovery was 98%.

The strike-through time of the first layer was 3.1 sec.

The second layer had basis weight 60 gsm and comprised endless filaments having a concentric core/sheath cross section, wherein the core consisted of PET (Invista 5520) and formed 70% by weight of the filament and the sheath consisted of coPET (Trevira RT5023) and formed 30% by weight of the filament. The mean filament diameter was 32.5 microns.

The filaments were left untreated.

The thickness of the second layer was 1.8 mm, the calculated void volume was 97.57% and the recovery was 99%.

The strike-through time of the second layer was 3.5 sec.

The third layer had basis weight 120 gsm and comprised curly crosslinked cellulose fibres. The mean fibre diameter was 25 microns.

The fibres were left untreated.

The thickness of the third layer was 2.8 mm and the recovery was 95%.

The strike-through time of the second layer was 2.3 sec.

Superabsorbent particles (EVONIC SAP FAVOR SXM 9170) in the amount of 333 g/m², the majority of which have the size within the range of 300 to 850 microns, were evenly distributed on the first layer and partially within the first layer.

The second layer was placed on the superabsorbent layer.

Each of the above-described examples has been arranged within a wrap to prepare a testing sample. The wrap consisted of a nonwoven fabric of the SMS type (spunbond/meltblown/spunbond) having basis weight of 13 gsm, and the wrap surrounded the nonwoven fabric structure. Such a sample was then tested using a device for measuring diaper absorption capacity (by Hytec). Each sample was laid in the device on a foil such that the second layer was above the first layer. Then, each sample was exposed to 70 ml of physiological (0.9%) NaCl solution 4 times with a 10-minute waiting time between the individual 70 ml liquid strikes; the absorption time was measured after each of the liquid strikes.

Table 1 shows the results for the individual examples. The absorption time of the last 5 ml is usually considerably longer than the absorption time of the first 5 ml. The last column contains the time elapsed between the first liquid exposition and the moment, in which all but the approximately last 5 ml of the liquid/physiological solution is absorbed. This data may be considered as most relevant for assessing the duration of exposition of a wearer's skin to the liquid after a strike of liquid. The nonwoven fabric structure according to the invention is usually arranged within an absorbent article such that there may be one or several layers arranged between the wearer's skin and the second (or third) layer of the nonwoven fabric structure. The moment, when there is only 5 ml of remaining/not yet absorbed liquid on the upper surface of the nonwoven fabric structure, corresponds to the situation, when for example a modern soft bulky topsheet covering the nonwoven fabric structure starts to exhibit a dry surface.

TABLE 1

| | Average of | Average of | Average of | Average of | |
|---|---|---|---|---|---|
| | absorption | absorption | absorption | absorption | |
| | time 1 | time 2 | time 3 | time 4 | |
| | (1st STT) | (2nd STT) | (3rd STT) | (4th STT) | 1st STT - |
| Example | sec/70 ml | sec/70 ml | sec/70 ml | sec/70 ml | till last 5 ml |
| 0 | 101 | 189 | 248 | 528 | 23 |
| 1 | 27 | 57 | 72 | 86 | 8 |
| 2 | 22 | 51 | 64 | 71 | 9 |
| 3 | 42 | 80 | 108 | 143 | 11 |
| 4 | 41 | 70 | 88 | 103 | 10 |
| 5 | 32 | 51 | 58 | 68 | 8 |
| 6 | 54 | 143 | 162 | 236 | 11 |
| 7 | 47 | 72 | 93 | 105 | 9 |
| 8 | 44 | 100 | 121 | 156 | 8 |
| 9 | 35 | 66 | 90 | 119 | 7 |
| 10 | 5 | 23 | 26 | 29 | 5 |
| 11 | 7 | 21 | 26 | 29 | 7 |
| 12 | 6 | 21 | 27 | 34 | 6 |
| 13 | 11 | 38 | 43 | 46 | 11 |
| 14 | 7 | 27 | 33 | 39 | 7 |
| 15 | 7 | 21 | 26 | 29 | 7 |

The above data shows that the nonwoven fabric structure according to the invention has an excellent (i.e. very short) strike-through time at the first, second, third and fourth liquid insult. Consequently, the skin of a wearer of an absorbent article comprising the inventive nonwoven fabric structure, will be in contact with the liquid for a shorter time and also the risk of leakage will be significantly reduced.

Testing Methodology

The “Basis weight” of a nonwoven web is measured according to the European standard test EN ISO 9073-1:1989 (conforms to WSP 130.1). There are 10 nonwoven web layers used for measurement, sample area size is 10×10 cm2.

The “Thickness” or “Calliper” of the nonwoven material is measured according to the European standard test EN ISO 9073-2:1995 (conforms to WSP 120.6) with the following modification:

1. The material shall be measured on a sample taken from production without being exposed to higher strength forces or spending more than a day under pressure (for example on a product roll), otherwise before measurement the material must lie freely on a surface for at least 24 hours.

2. The overall weight of the upper arm of the machine including added weight is 130 g.

“Median fibre diameter” in a layer is expressed in SI units—micrometers (μm) or nanometers (nm). To determine the median, it is necessary to take a sample of the nonwoven fabric from at least three locations at least 5 cm away from each other. In each sample, it is necessary to measure the diameter of at least 50 individual fibres for each observed layer. It is possible to use, for example, an optical or electronic microscope (depending on the diameter of the measured fibres). In the event that the diameter of fibres in one sample varies significantly from the other two, it is necessary to discard the entire sample and to prepare a new one.

In the case of round fibres, the diameter is measured as a diameter of the cross-section of the fibres. In the event of any other shape of the fibre (e.g. hollow fibre or trilobal fibre), the cross-section surface shall be determined for each measured fibre and recalculated for a circle with same surface area. The diameter of this theoretical circle is the diameter of the fibre.

The measured values for each layer composed of all three samples are consolidated into a single set of values from which the median is subsequently determined. It applies that at least 50% of the fibres have a diameter less than or equal to the value of the median and at least 50% of the fibres have a diameter greater than or equal to the median. To identify the median of the given sample set of values, it is sufficient to arrange the values according to size and to take the value found in the middle of the list. In the event that the sample set has an even number of items, usually the median is determined as the arithmetic mean of the values in locations N/2 and N/2+1.

The “void volume” herein refers to the total amount of void space in a material relative to the bulk volume occupied by the material.

The bulk volume of the material is equal to the bulk volume of the nonwoven and can be calculated from fabric thickness (calliper) using the following equation:

bulk volume (m³)=(calculated thickness, i.e. calliper (m))*1 (m)*1 (m)

The total amount of void space in a material can be calculated using the equation:

void space=bulk volume (m³)−mass volume (m³)

The mass volume can be calculated using the equation:

mass volume (m³)=(weight in kilograms based on basis weight (kg))/mass density (kg/m³)

Where the mass density can be calculated from a known composition or measurement according to the norm ISO 1183-3:1999.

So the void volume of a layer comprising endless filaments can be calculated using the equation:

Void volume (%)=[1−(volume of filaments in 1 m² nonwoven fabric layer/volume of 1 m² nonwoven fabric layer)]*100%

Thus, for single component filaments:

Void volume (%)=[1−(basis weight (g/m²)/thickness (mm))/mass density (kg/m³)]*100%

Of course, when multi-component filaments are considered, wherein the components differ in density, the volume of filaments within a square meter of nonwoven fabric (NT) must be calculated accordingly.

As may be deduced from the above calculation, the superabsorbent particles eventually fallen into some of the pores/void volumes of the non-woven layer of filaments are not taken into account in the calculated void volume.

The most precise determination, whether a product falls within the scope of the independent claim regarding the calculated void volume, is based on data regarding the basis weight of the first layer, its thickness, fibre thickness and fibre material.

In case that data about the basis weight of the first layer of filaments without superabsorbent (and eventually including glue) are not available, for example the following procedure can be used to estimate the properties of the nonwoven fabric:
a sample of the nonwoven fabric structure is carefully separated to obtain a first layer and a second layer and superabsorbent particles are carefully knocked out from both layers. When free superabsorbent has been removed from the fabric, the following procedure for the removal of attached particles can be used:
the sample is immersed into clean water and fully soaked (after 30 min of immersing the sample is still under water)
the sample is removed from water, left to drip off and inserted into a centrifuge in such a way, that the surface of the fabric is located perpendicularly to the action of the centrifugal force. Particles of soaked superabsorbent are heavy and are released from the fabric by action of the centrifugal force. A person skilled in the art can correctly choose application of a suitable rotation speed to allow release of the particles and avoid damaging of the fabric.
the fabric is checked and if superabsorbent articles are found, the procedure is repeated in such a way that the fabric is inserted into the centrifuge perpendicularly to the action of the centrifugal force such that the outer side is directed inwards during the first centrifugation.
the procedure is repeated until the entire superabsorbent is removed from the fabric. The fabric is left to dry in an oven with a temperature up to 40° C. (until a constant weight is reached). Then it is possible to measure the basis weight, average fibre diameter or mass density of the fibres. The sample can also be used to estimate elasticity or compressibility, in that case it is necessary to consider suitability based on damage of the surface of the fabric during disintegration of the fabric structure.

The “recovery” of the bulkiness after the application of pressure herein refers to the ratio of the thickness of the fabric after it is freed from a load to the original thickness of the fabric. The thickness of the fabric is measured according to the EN ISO 9073-2:1995 using a preload force of 0.5 kPa). The recovery measurement procedure consists of following steps:
1. Prepare fabric samples measuring 10×10 cm
2. Measure the thickness of 1 piece of fabric
3. Measure the thickness of a pile of 5 pieces of fabric using a preload force of 0.5 kPa (Ts)
4. Load the pile of 5 fabric sheets on to a thickness meter (2.5 kPa) for 5 minutes
5. Release the weight and wait for 5 minutes
6. Measure the thickness of pile of the 5 fabrics using a preload force of 0.5 kPa (Tr)
7. Calculate the recovery according to the following equation:

Recovery=Tr/Ts (no unit)
Ts=thickness of fresh sample
Tr=thickness of recovered sample

The “compressibility” herein refers to the distance in mm by which the nonwoven is compressed by the load defined in the “resilience” measurement. It can be also be calculated as resilience (no unit)*thickness (mm). The “resilience” of a nonwoven is measured according to the European standard test EN ISO 964-1 with the following modification:
1. The thickness of fabric is measured.
2. A pile of fabric samples is prepared so that the total thickness is at least 4 mm, optimally as close to 5 mm as possible in total. The pile of fabrics contains at least 1 piece of fabric.
3. The thickness of the pile of fabric samples is measured
4. A force of 5N with loading speed of 5 mm/min is applied to the pile of nonwoven samples
5. The distance of the clamp movement is measured
6. Resilience is calculated according to the equation:

R (no unit)=T1(mm)/T0(mm)

Or

R (%)=T1(mm)/T0(mm)*100%

T1=distance of the clamp movement under the load 5N [mm]=how much was the pile of fabrics compressed

T0=thickness (acc. EN ISO 9073-2:1995 using the preload force of 1.06N) [mm]

The “degree of crimping” is measured according to ASTM D-3937-82 with the following modification:

1. the used unit of measurement is “crimps/cm”

Setting the degree of crimping in a bonded layer is an issue since single fibres are bonded to each other and it is not possible to remove one of them from the composition (without the danger of affecting the original crimp level) and to measure the crimp value and the fibre length. For the purpose of this invention, the following estimation may be used:
1) A picture of the assessed layer is provided in such a magnification that the fibres can be well seen
2) One single fibre is chosen and its path through the picture or at least part of the picture is marked
3) The length of the marked fibre in the picture is measured
4) The number of crimps in the measured length is counted.
In contrast to the measurement of individual fibres, it is not possible to place the fibre in such a way that all the crimps can be seen clearly and then counted in a repeating sequence. In a bonded structure, some parts may be masked in the z direction, some parts may be masked by other fibres; some parts may be masked by bonding. Each fibre turn shall be counted as half a crimp. Also, a change from sharp to blurry on one fibre shall be counted as half a crimp
5) The number of crimps/cm is calculated

It has to be kept in mind that the value is calculated from a 2D picture of a 3D object and that the length of the fibre in the z direction is not covered. The real length of the fibre would probably be higher. Also, a 2D picture can mask certain crimps on the fibre, especially in the vicinity of a bonding point. Nevertheless, it is assumed that the described calculation can provide a relevant estimate of fibre crimping.

The “resilience” of a nonwoven is measured according to the European standard test EN ISO 964-1 with the following modification:

1. A preload force of 1.06N is applied to the nonwoven sample.

2. A force of 5N with a loading speed of 5 mm/min. is applied to the nonwoven sample.

Resilience is then calculated using the following equation:

R (no unit)=T1(mm)/T0(mm)

Or

R (%)=T1(mm)/T0(mm)*100%

T1=distance of the clamp movement under the load 5N [mm]=how much was the pile of fabrics compressed

T0=thickness (acc. EN ISO 9073-2:1995 using the preload force 1.06N) [mm]

R=resilience

T₁=distance between the clamps under a load of 5N [mm]

T₀=thickness (acc. EN ISO 9073-2:1995) [mm]

The “Bulk density” of a nonwoven material is calculated using the following equation:

ρ_{b}=bulk density [kg/m³]=basis weight (g/m2)/fabric thickness (mm)

BW=basis weight (acc. EN ISO 9073-1:1989) [g/m²]

T=thickness (acc. EN ISO 9073-2:1995) [mm]

The bulk density of one layer in a composite:
1) Using an optical method, the thickness of a single layer in the cross-section of a nonwoven is measured. The number of samples is at least 10 and the number is set so that the corrected sample standard deviation s shall be smaller than 30% of the average value (v is below 30%)
2) The basis weight is measured
   a. The production value is taken
   b. To obtain an approximate value, it is possible to do the following:
      i. A sample of a known surface area is taken
      ii. The layers are carefully separated from each other, or the fibres from the layers are separated out,
      iii. The weight of the separated layers and the fibres from them are measured.
      iv. The basis weight is calculated from the known surface area and the weight of layer.
      v. The number of samples is at least 10 and the number is set so that the corrected sample standard deviation s is less than 20% of the average value (v is below 20%)

The corrected sample standard deviation shall be calculated using following formula:

$$s = \sqrt{\frac{1}{N - 1} {\sum }_{i = 1}^{N} {( {x}_{i} - \overline{x} )}^{2}} . \\ v = \frac{s}{x} \cdot 100 ( \% )$$

Where:

N— number of samples

xi—single measured value

x—average measured value

The “length of the filament to the length of the fabric ratio” can be measured in three different ways:

a) The length of the filaments is measured by stretching them out so that they extend along a line without exhibiting crimps. The method is the better the lower the bonding is.

b) In a fabric bonded to a given level, it is not possible to use method a) to measure the length of the filaments, so that the following estimation may be used:
a. A picture of the assessed layer is provided in such a magnification that the fibers can be well seen
b. One single fiber is chosen and its path through the picture or at least through part of the picture is marked out
c. The length of fiber marked out on the picture is measured to estimate its real length
d. The length of the fabric, where the fiber is marked out is measured
e. The estimated length of the filament to the length of the fabric ratio (percentage) is calculated for at least 20 fibers.

c) In a fabric using the “Method for determining geometric fiber statistics for a nonwoven material”, where:
a. The geometric representation of the fabric for analysis measures 8 mm in MD and 8 mm in CD, maintaining the full thickness of the sample in z-direction.
b. Only the fibers, that enter the cropped sample volume on one side and leave it at opposite side are relevant for the measurement
c. At least 20 filaments have to be measured
d. The length of the filament to the length of the fabric ratio (percentage) is calculated

1. A nonwoven fabric structure comprising a first layer, a second layer and superabsorbent particles, said first layer comprising endless filaments,
which comprise at least a first polymeric material (A) and a second polymeric material (B) having its melting point lower than the first polymeric material (A),
wherein the second polymeric material (B) extends in the longitudinal direction of the filament and forms at least a part of the surface of the filament and
the first layer contains filament-to-filament bonds formed of the second polymeric material (B),
a calculated void volume between the filaments of the first layer forms at least 65% of the volume of the first layer, wherein the calculated void volume between the filaments of the first layer=[1−(volume of the mass of filaments in 1 m² of the first layer/volume of 1 m² of the first layer)]*100%,
the second layer comprising endless filaments,
wherein the superabsorbent particles are arranged at least between the first layer and the second layer and within some of the voids of the first layer and/or the second layer.

2. The nonwoven fabric structure according to claim 1, wherein all components of the filaments of the first layer are arranged across the cross-section of the filament in a configuration not supporting crimping.

3. The nonwoven fabric structure according to claim 1, wherein
at least 20% of the filaments of the first layer have a length of filament to length of fabric ratio higher than 1.2:1, and/or
at least 10% of the filaments of the first layer have a ‘length of filament to length of fabric ratio’ higher than 1.5:1, and/or
at least 10% of the filaments of the first layer have a ‘length of filament to length of fabric ratio’ lower than 2.5:1.

4. The nonwoven fabric structure according to claim 1, wherein the calculated void volume between the filaments of the first layer forms at least 75% of the volume of the first layer preferably at least 80%; more preferably at least 84%, more preferably at least 86%, more preferably of at least 88%, more preferably at least 90%, most preferably at least 93% of the volume of the first layer.

5. The nonwoven fabric structure according to claim 1, wherein the first polymeric material (A) and/or the second polymeric material (B) of the filaments of the first layer consists of or comprises as the majority component polymeric material selected from the group consisting of polyesters, polyolefins, polylactic acid, polyester copolymers, polylactide copolymers and blends thereof; and the first polymeric material (A) is different from the second polymeric material (B).

6. The nonwoven fabric structure according to claim 1, wherein the filaments of the first layer and or of the second layer have a core/sheath structure, wherein the first polymeric material (A) forms the core and the second polymeric material (B) forms the sheath.

7. The nonwoven fabric structure according to claim 1, wherein the first layer and/or the second layer has a basis weight of at least 5 gsm, preferably of at least 10 gsm, more preferably of at least 20 gsm, more preferably of at least 30 gsm, with advantage of at least 40 gsm and preferably not greater than 200 gsm, preferably not greater than 150 gsm, preferably not greater than 100 gsm, most preferably not greater than 80 gsm.

8. The nonwoven fabric structure according to claim 1, wherein the filaments of the first layer and/or of the second layer have a median fibre diameter of at least 5 microns; preferably at least 10 microns; preferably at least 15 microns; most preferably at least 20 microns, and at most 50 microns; preferably at most 40 microns; most preferably at most 35 microns.

9. The nonwoven fabric structure according to claim 1, wherein
the endless filaments of the second layer comprise at least the first polymeric material (A) and the second polymeric material (B), which extends in the longitudinal direction of the filaments and forms at least a part of the surface of the filaments and
the second layer contains filament-to-filament bonds formed by the second polymeric material (B).

10. The nonwoven fabric structure according to claim 1, wherein the filaments of the second layer have an eccentric core/sheath structure.

11. The nonwoven fabric structure according to claim 9, wherein the filaments of the second layer are crimped filaments exhibiting at least 3 crimps/cm.

12. The nonwoven fabric structure according to claim 1, wherein it further comprises a third layer of endless filaments arranged adjacent the second layer at the side facing away from the first layer, the endless filaments of the second and third layer being both crimped filaments exhibiting at least 3 crimps/cm.

13. The nonwoven fabric structure according to claim 1, wherein the filaments of the first layer and the filaments of the second layer have hydrophilic surface.

14. The nonwoven fabric structure according to claim 1, wherein the first layer is more hydrophilic than the second layer.

15. The nonwoven fabric structure according to claim 1, wherein the superabsorbent particles are superabsorbent polymer particles,

16. The nonwoven fabric structure according to claim 1, wherein the superabsorbent particles contain crosslinked, partly neutralized polyacrylic acid and/or sodium polyacrylate.

17. The nonwoven fabric structure according to claim 1, wherein at least 50% by weight, more preferably at least 65% by weight of the superabsorbent particles has the size within the range of 300 to 850 microns.

18. The nonwoven fabric structure according to claim 1, wherein it contains at least 200 g, more preferably at least 300 g, more preferably at least 400 g of superabsorbent particles per m2, wherein it contains less than 800 g, more preferably less 700 g, most preferably less than 600 g of superabsorbent particles per m2.

19. An absorbent hygienic product comprising a topsheet, a backsheet and the nonwoven fabric structure according to claim 1 arranged between the topsheet and the backsheet.

20. The absorbent hygiene product according to claim 19, wherein the first layer is arranged closer to the backsheet than the second layer.
